(19) United States
(12) Patent Application Publication
(10) Pub. No.: US 2006/0141631 A1
(43) Pub. Date: Jun. 29, 2006

(54) Quantitation of biological molecules

(76) Inventors: Pavel V. Bondarenko, Thousand Oaks, CA (US); Thomas A. Shaler, Fremont, CA (US); Dirk H. Chelius, Camarillo, CA (US)

Correspondence Address: FISH & RICHARDSON P.C., PO BOX 1022, MINNEAPOLIS, MN, 55440-1022, US

(21) Appl. No.: 10/511,490

(22) PCT Filed: Apr. 15, 2003
(86) PCT No.: PCT/US03/11870

§ 371 Date: Oct. 14, 2005

Related U.S. Application Data
(60) Provisional application No. 60/373,007, filed on Apr. 15, 2002.

(51) Int. Cl. G01N 33/48

(52) U.S. Cl. 436/86

(57) ABSTRACT

Methods and apparatus, including computer program products, for quantifying peptides in a peptide mixture. A peptide mixture containing a plurality of peptides is received. One or more peptides are separated from the peptide mixture over a period of time. One or more of the peptides separated at a particular time are subjected to mass-to-charge analysis and an abundance of one or more of the mass analyzed peptides is calculated. A relative quantity for the one or more mass analyzed peptides is calculated by comparing the calculated abundance of the peptides with an abundance of one or more peptides in a reference sample that is external to the first peptide mixture. The techniques can be applied to arbitrary peptides, without requiring the use of differential mass labeling, and can be applied to other biological molecules, such as nucleic acids and small molecules.

### CROSS REFERENCE TO RELATED APPLICATIONS

This application claims the benefit of U.S. Provisional Application No. 60/373,007, filed Apr. 15, 2002, which is incorporated by reference herein.

### TECHNICAL FIELD

This invention relates to analytical techniques for identification and quantification of polypeptides.

### BACKGROUND

For a number of years, two dimensional gel electrophoresis (2D GE) has been the standard method for separation and quantitation of protein mixtures. Binding different dyes to the proteins (staining), for example Coomassie blue, or using radioactive labels, for example ³²p, makes it possible to visualize protein spots on the gels. After scanning the gels, densitometry has been used to measure the “darkness” of the spots, and obtain quantitative information. In the 1990's, mass spectrometry (MS) became a popular tool for identification of proteins after their in-gel digestion. Although widely used, 2D GE-MS has limitations when dealing with very large or small proteins, proteins at the extremes of pI scale, membrane and low abundance proteins. The amount of attached dye is not linearly proportional to the concentration, so reliability of this quantitation is still questionable. In addition, it can take two days or more to run a single 2D gel, and staining and destaining before mass spectrometry takes additional time. Radiography is also a very tedious procedure. Finally, excising the gel spots, digesting proteins, extracting the proteolytic products and analyzing each individual spot by mass spectrometry are also time- and labor-intensive steps.

Quantitation of peptide and protein mixtures by mass spectrometry has been a challenging analytical problem, largely because of ionization suppression among co-eluting species. To address these challenges, stable isotope-labeled peptides have been employed as internal standards for mass spectrometry. These compounds make attractive standards, because, while they differ in mass, their chemical and physical properties, such as chromatographic retention time and ionization efficiency, are similar to those of their unlabeled counterparts. These techniques avoid the need for 2D GE and densitometry, but give rise to an entirely different set of challenges. It can be difficult to achieve complete substitution of a natural isotope (e.g., ¹⁶O) with a rare stable isotope (e.g., ¹⁸O) to create a standard protein mixture, which results in a large number of protein molecules in which only a fraction of the intended atoms is substituted. Rare isotope labeling reagents are also expensive, and working with such reagents requires additional safety measures and skills.

### SUMMARY

The invention provides techniques for relatively quantifying molecules in biological mixtures. In general, in one aspect, the invention provides methods and apparatus, including computer program products, implementing techniques for quantifying peptides in a peptide mixture. The techniques include receiving a first peptide mixture containing a plurality of peptides, separating one or more of the plurality of peptides of the first peptide mixture over a period of time, mass-to-charge analyzing one or more of the separated peptides of the first peptide mixture at a particular time in the period of time, calculating an abundance of one or more of the mass analyzed peptides of the first peptide mixture, and calculating a relative quantity for the one or more mass analyzed peptides of the first peptide mixture by comparing the calculated abundance of the one or more mass analyzed peptides of the first peptide mixture with an abundance of one or more peptides in a reference sample. The reference sample is external to the first peptide mixture.

Particular embodiments can include one or more of the following features. Receiving a first peptide mixture containing a plurality of peptides can include digesting a first polypeptide sample to generate the first peptide mixture. The techniques can include preparing the reference sample by digesting a second polypeptide sample, separating one or more peptides from the digested second polypeptide sample, mass analyzing the separated peptides from the digested second polypeptide sample, and calculating an abundance of one or more of the mass analyzed peptides from the second polypeptide sample. Calculating a relative quantity for the one or more mass analyzed peptides of the first peptide mixture can include comparing the calculated abundance of the one or more mass analyzed peptides of the first peptide mixture with the calculated abundance of one or more corresponding mass analyzed peptides from the second polypeptide sample. Separating one or more peptides can include separating the one or more peptides by liquid chromatography.

Separating one or more peptides can include isolating a liquid chromatography eluent at the particular time, and mass analyzing one or more of the separated peptides of the first peptide mixture can include mass analyzing one or more peptides in the isolated eluent.

The techniques can include identifying one or more peptides of the first peptide mixture. Identifying one or more peptides of the first peptide mixture can include identifying one or more of the separated peptides based on mass analysis information. Mass analyzing one or more of the separated peptides can include fragmenting an ion derived from a peptide of the one or more separated peptides and mass analyzing fragments of the ion. Identifying one or more peptides in the first sample can include searching a sequence database based on mass analysis information for the fragments.

Calculating an abundance of one or more of the mass analyzed peptides can include reconstructing a chromatogram peak for a peptide based on mass analysis information for the peptide. Calculating an abundance for a peptide can include calculating an abundance for a peptide based on a reconstructed chromatogram peak area for the peptide. Calculating the abundance for a peptide can include calculating an abundance for a peptide using only chromatogram peaks located within a threshold distance in the reconstructed chromatogram of the particular time.

Calculating a relative quantity for the one or more mass analyzed peptides can include comparing an abundance calculated by reconstructing a chromatogram peak area for a peptide of the first peptide mixture with an abundance calculated by reconstructing a chromatogram peak area for a peptide in the reference sample.

The techniques can include normalizing the calculated abundance of the one or more mass analyzed peptides of the first peptide mixture. Normalizing the calculated abundance can include normalizing the calculated abundance based on an internal standard including one or more peptides added to the first polypeptide sample. Normalizing the calculated abundance can include normalizing the calculated abundance based on an external standard including one or more peptides.

The techniques can include identifying a plurality of peptides of the first peptide mixture based on the mass analyzing, wherein calculating a relative quantity for the one or more mass analyzed peptides comprises calculating a relative quantity for each of the identified peptides. Calculated abundances for each of the identified peptides can be normalized by calculating a correction factor based on reconstructed chromatogram peak areas for a set of peptides in the first peptide mixture, where each peptide in the set of peptides has constant chromatogram peak areas over a plurality of experiments, and applying the correction factor to the calculated abundance for each of the identified peptides.

The mass analyzing and calculating steps can be performed to identify and calculate relative quantities for every peptide in the first peptide mixture in a single automated experiment.

The one or more of the separated peptides that are subjected to the mass-to-charge analyzing and calculating steps can be naturally occurring peptides. The one or more peptides in the reference sample can be naturally occurring peptides. Mass-to-charge analyzing one or more of the separated peptides and calculating an abundance of one or more of the mass analyzed peptides can include mass-to-charge analyzing and calculating an abundance for one or more arbitrary peptides of the first peptide mixture. The techniques can be implemented such that the separating, mass-to-charge analyzing, and calculating steps are not constrained to a particular amino acid composition of the subject peptides.

In general, in another aspect, the invention provides methods and apparatus, including computer program products, implementing techniques for quantifying quantifying one or more peptides in a mixture. The techniques include digesting a protein sample to generate a mixture of peptides, separating one or more peptides of the mixture of peptides using liquid chromatography, mass analyzing one or more of the separated peptides, identifying one or more of the mass analyzed peptides based on mass spectra for the peptides, calculating chromatogram peak areas for the identified peptides, calculating chromatogram peak areas for one or more proteins corresponding to the identified peptides based on the calculated peak areas for the corresponding peptides, normalizing the chromatogram peak area for the protein based on a chromatogram peak area for an internal standard, and determining a relative quantity for a protein of the one or more of the proteins by comparing the normalized chromatogram peak area for the protein to a chromatogram peak area for a corresponding protein in a reference sample.

In general, in still another aspect, the invention features methods and apparatus, including computer program products, implementing techniques for quantifying one or more compounds in a biological sample. The techniques include receiving a biological sample containing a plurality of compounds, separating one or more of the plurality of compounds of the biological sample over a period of time, mass-to-charge analyzing one or more of the separated compounds of the biological sample at a particular time in the period of time, calculating an abundance of one or more of the mass analyzed compounds of the biological sample, and calculating a relative quantity for the one or more mass analyzed compounds of the biological sample by comparing the calculated abundance of the one or more mass analyzed compounds of the biological sample with an abundance of one or more compounds in a reference sample, the reference sample being external to the biological sample.

The invention can be implemented to achieve one or more of the following advantages. Using the disclosed techniques, the relative abundance of proteins in, for example, a group of cells treated by drug, nutrient, toxin, etc. can be compared with proteins from a control group of cells to find those proteins which are over-expressed or under-expressed under the influence of the reagent. The techniques can be implemented to search for and quantify disease markers or drug targets, and/or to screen potential drugs. The described techniques can be implemented to avoid the limitations in accessing proteins at the extremes of molecular weight and pI scale that are present in prior gel electrophoresis methods. The techniques are not limited by the content of the sample or the nature of the polypeptide, specific amino acids, etc, and can be performed on naturally-occurring proteins and peptides. No labor-intensive and time-consuming labeling of samples is needed prior to analysis. Likewise, no expensive reagents are required to create an internal standard, as in isotope-coded affinity tag (ICAT) or similar methods. The techniques are not limited to proteins that contain particular amino acids (such as cysteine). An unlimited number of samples can be compared. Each sample is analyzed in a separate experiment, and each can be referenced to the same reference sample if desired. The sample and the reference sample experiments are distinct experiments. Using two-dimensional liquid chromatographic techniques in combination with tandem mass spectrometry makes it possible to identify and quantify proteins incorporating unknown modifications, as well different proteins having the same mass.

Complete separation of the peptides is not required; rather, even a partial separation of peptides can be sufficient for quantitation using the techniques described herein. The techniques can be implemented to identify all proteins in a mixture in one automated step.

The details of one or more embodiments of the invention are set forth in the accompanying drawings and the description below. Unless otherwise defined, all technical and scientific terms used herein have the meaning commonly understood by one of ordinary skill in the art to which this invention belongs. All publications, patent applications, patents, and other references mentioned herein are incorporated by reference in their entirety. In case of conflict, the present specification, including definitions, will control. Other features and advantages of the invention will become apparent from the description, the drawings, and the claims.

### BRIEF DESCRIPTION OF THE DRAWINGS

FIG. 1 is a flow diagram illustrating one implementation of a method for quantifying peptides in a mixture of peptides according to one aspect of the invention.

FIG. 2 is a schematic diagram illustrating a system operable to quantify peptides in a mixture of peptides according to one aspect of the invention.

FIG. 3 is a more detailed flow diagram illustrating one implementation of a method for quantifying peptides in a mixture of peptides according to one aspect of the invention.

FIG. 4 illustrates a typical ion chromatogram of a five-protein mixture, provided by one implementation of one aspect of the invention (the sequence “TGPNLHGLFGR” is SEQ ID NO:25).

FIG. 5A and 5B illustrate a typical fragmentation mass spectrum and its interpretation, provided by one implementation of one aspect of the invention (the sequence “TGPNLHGLFGR” is SEQ ID NO:25).

FIG. 6 is an example of a chromatographic peak area reconstructed according to one implementation of one aspect of the invention (the sequence “TGPNLHGLFGR” is SEQ ID NO:25).

FIG. 7 illustrates eight reconstructed chromatograms for ions of a myoglobin peptide and an albumin peptide according to one aspect of the invention.

FIG. 8 illustrates a calibration curve for myoglobin digest, according to one aspect of the invention.

FIG. 9 illustrates a calibration curve for cytochrome C, according to one aspect of the invention.

FIGS. 10(a) and (b) illustrate the base peak ion chromatograns of human plasma digests spiked with 250 and 500 fmol myoglobin, respectively, according to one aspect of the invention.

FIGS. 10(c) and (d) illustrate the reconstructed ion chromatograms of identified myoglobin peptides, in human plasma spiked with 250 and 500 fmol myoglobin, respectively, according to one aspect of the current invention.

FIG. 11 illustrates the changes of combined chromatographic peak area for different amounts of myoglobin injected, according to one aspect of the current invention.

Like reference numbers and designations in the various drawings indicate like elements.

### DETAILED DESCRIPTION

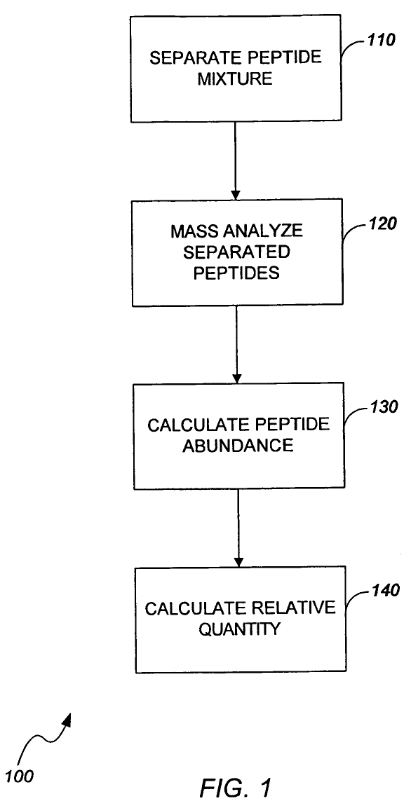

The invention provides methods and apparatus, including computer program products, for quantifying peptides and proteins. Referring to FIG. 1, a method 100 of quantifying peptides in a mixture of peptides according to one aspect of the invention begins with the separation of a collection of peptides derived from a protein sample (step 110). The separated peptides are subjected to mass analysis (step 120). The separation and mass analysis information is used to calculate an abundance for each of one or more peptides in the mixture (step 130). The relative quantity of a given peptide is calculated by comparing the calculated abundance for the peptide with an abundance calculated for a reference sample (step 140). The reference sample abundance can be calculated by performing steps 110 through 130 with a reference sample, as will be described in more detail below. The method 100 can be repeated with any number of samples, such that an arbitrary (i.e., potentially unlimited) number of samples can be compared with each other and with the reference sample. Each sample is analyzed in a separate experiment, and each can be referenced to the same reference sample if desired. The sample and the reference sample experiments are distinct experiments.

As used in this specification, a peptide or polypeptide is a polymeric molecule containing two or more amino acids joined by peptide (amide) bonds. As used in this specification, a peptide typically represents a subunit of a parent protein or polypeptide, such as a fragment produced by proteolytic cleavage using enzymes, or using chemical or physical means. Peptides and polypeptides can be naturally occurring (e.g., proteins or fragments thereof) or of synthetic nature. Polypeptides can also consist of a combination of naturally occurring amino acids and non-naturally occurring amino acids. Peptides and polypeptides can be derived from any source, such as animals (e.g., humans), plants, fungi, bacteria, and/or viruses, and can be obtained from cell samples, tissue samples, organs, bodily fluids, or environmental samples, such as soil, water, and air samples. Polypeptides can be membrane-associated (i.e., spanning a lipid bilayer or adsorbed to the surface of a lipid bilayer). Membrane-associated polypeptides can be associated with, for example, plasma membranes, cell walls, organelle membranes, and viral capsids. Polypeptides can be cytoplasmic or organeller. Polypeptides can be extracellular, being found interstitially or in bodily fluids (e.g., plasma, and spinal fluid). Polypeptides can be biological catalysts, transporters or carriers for a variety of molecules, receptors for intercellular and intracellular signaling, hormones, and structural elements of cells, tissues and organs. Some polypeptides are tumor markers. As used in this specification a protein is a polypeptide.

It is noted that it is common in the field of mass spectrometry to speak in abbreviated fashion in terms of “mass” of ions, although it would be more precise to speak of the mass-to-charge ratio of ions, which is what is really being measured. For convenience, this specification adopts the common practice, and frequently uses the term “mass” to mean mass-to-charge ratios or quantities mathematically derived from those mentioned mass-to-charge ratios.

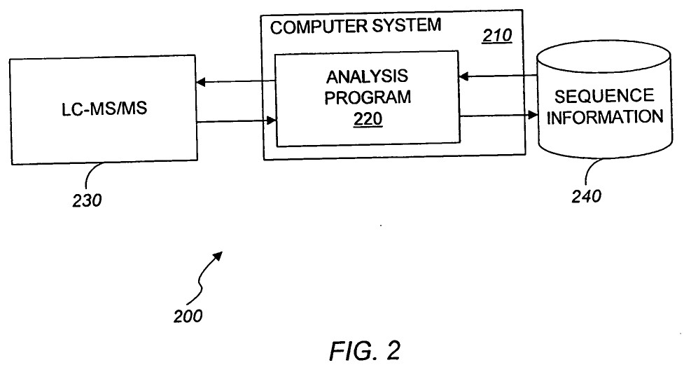

FIG. 2 illustrates one implementation of a system 200 for quantifying peptides in a mixture of peptides according to one aspect of the invention. System 200 includes a general-purpose programmable digital computer system 210 of conventional construction, which can include a memory and one or more processors running an analysis program 220. Computer system 210 has access to a source of mass spectral data 230, which can be a mass spectrometer, such as an LC-MS/MS mass spectrometer. Alternatively, or in addition, mass spectral data can be retrieved from a database accessible to computer system 210. Computer system 210 is also coupled to a source of sequence information 240, such as a public database of amino acid or nucleotide sequence information. System 200 can also include input devices devices, such as a keyboard and/or mouse, and output devices such as a display monitor, as well as conventional communications hardware and software by which computer system 210 can be connected to other computer systems (or to mass analyzer 230 and/or database 240), such as over a network.

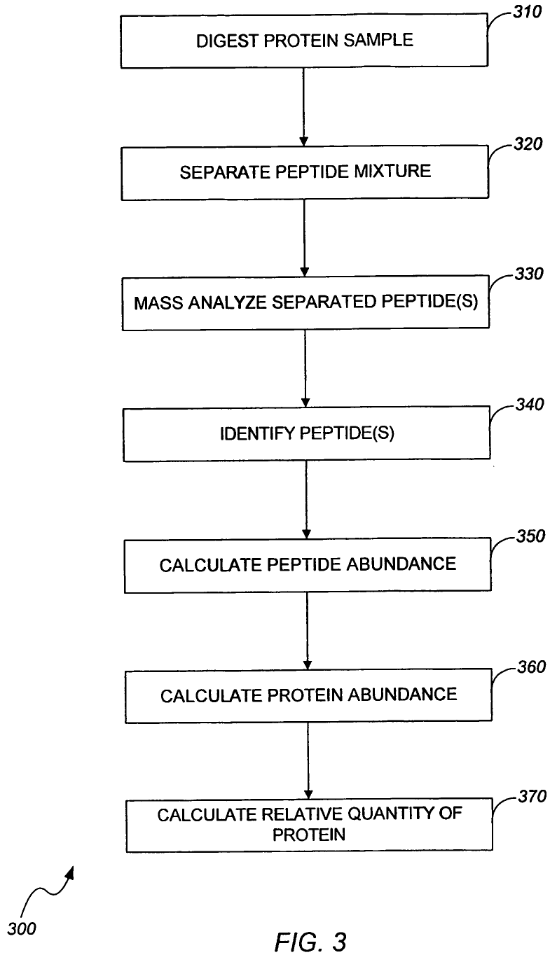

FIG. 3 illustrates one implementation of a method 300 according to one aspect of the invention in more detail. An experimental sample of one or more proteins to be quantified relative to a reference sample is digested to generate a mixture of peptides (step 310). The sample can be a simple mixture including only one or two proteins, contained for example in gel electrophoresis spots; alternatively, the sample can be a more complex protein mixture - for example, a sample of proteins contained in human plasma. The sample can be derived from any source, such as animals (e.g., humans), plants, fungi, bacteria, and/or viruses, and can be obtained from cell samples, tissue samples, bodily fluids, or environmental samples, such as soil, water, and air samples. The quantity, and often the identity, of one or more proteins in the experimental sample will typically be unknown. The sample, including any added internal standard, can be digested enzymatically, using any of a variety of proteolytic enzymes using known techniques, or using known chemical or physical means.

The peptide mixture is separated (step 320). The mixture can be separated by a variety of known separation methods, including, but not limited to liquid chromatography, gas chromatography, electropheresis, and capillary electropheresis, either singularly or in combination. Particular conditions for the separation, including, for example, the type of media and column, solvents and flow rate, can be selected based on the particular experiment and on the separation desired. In one embodiment, the peptide mixture is separated using one dimensional liquid chromatography using a reversed-phase capillary column. If more complex separation is required, additional dimensions of liquid chromatography can be utilized, such as, two-dimensional liquid chromatography involving an initial separation on a strong cation exchange column, followed by a subsequent reversed-phase capillary column separation. In some cases, the separation can be performed to separate one or more individual peptides from the peptide mixture, although this is not required. However, even a partial separation of peptides can be sufficient for quantitation using the techniques described here, as the co-elution of two or more peptides during the separation should not interfere with the subsequent quantitation. This can be a significant advantage compared to other techniques, such as chromatographic separation with UV detection, where complete peak separation is required for quantitation. In general, a better separation will yield better ultimate results (i.e., better relative quantitation information).

The separated peptides are subjected to mass analysis (step 330). The separated peptides can be mass analyzed using any mass spectrometer with either MS and/or MS/MS capabilities that is capable of operating in conjunction with a liquid chromatograph to record MS and MS/MS data. In particular implementations, the mass spectrometer can be an ion trap, triple quadrupole, q-TOF, trap-TOF, FT-ICR, PSD TOF, TOF-TOF, or orbitrap spectrometer. A flull-scan mass spectrum is obtained for each peptide or combination of peptides separated in step 320—e.g., for each peak in the liquid chromatogram. An MS/MS spectrum is then obtained for each of one or more ions represented in the full-scan mass spectrum.

One or more of the separated peptides, and their corresponding proteins, are identified based on the tandem mass spectra generated for the peptides (step 340). Peptides and their corresponding proteins can be identified by correlating the experimental tandem mass spectra with theoretical fragmentation patterns derived from sequence information from a database, such as a publicly available database of nucleotide or amino acid sequences. For example, peptides and proteins can be identified by using commercially available database search engine software such as the TurboSEQUEST® protein identification software, available from Thermo Finnigan of San Jose, Calif., to compare tandem mass spectra obtained for the peptides with theoretical mass spectra determined for proteins (and fragments thereof) represented in a database of sequence information, such as the National Center for Biotechnology Information (NCBI), GenBank/GenPept, PIR, SWISS-PROT and PDB databases. Other database search engines, such as Mascot, ProFound, SpectrumMill, RADARS, Sonar software and the like, can also be used. Peptides and proteins can be identified using a closeness-of-fit or correlation score output by the search engine.

In one aspect of the invention, one or more of the separated peptides, and their corresponding proteins, are identified from full mass spectrum utilizing fourier transform and mass fingerprinting techniques. The one or more identified masses are then matched with data in a publicly available database.

Alternatively, peptides and proteins can be identified by partial or complete sequencing of the peptides in the separated peptides using de novo sequencing techniques, followed by localization of the resulting sequences in a publicly available database.

The mass spectra obtained in step 330 are then used to calculate the abundance of identified peptide ions (step 350). Ion abundance can be calculated as peak areas for each identified peptide by reconstructing the chromatogram for the corresponding identified peptide ion based on ion intensities measured in the mass spectra for the peptide. The peak area can be determined from the full mass spectra or the tandem mass spectra. Optionally, the reconstructed chromatogram and/or calculated peak areas can be graphically displayed to a user.

In one implementation, the abundance for a given peptide ion is calculated based on only the chromatographic peaks in the close vicinity from the time of identification, to avoid pseudo-peaks that are generated by species that are not proteolytic products of a particular protein, but that have similar m/z values. Thus, for example, only peaks within a predetermined threshold distance (i.e., time) from the time of identification can be used. The threshold can be defined according to the typical elution time of peptides in the particular area of the chromatagram, which depends on the flow rate, the separation techniques, the column utilized and the medium of separation, for example, and can range from a few seconds to several minutes. Removal of pseudo peaks can significantly improve the precision of peak area measurements. In one implementation, peak areas for identified peptide ions can be calculated using commercially-available software such as Xcalibur® software, available from Thermo Finnigan Corporation of San Jose, Calif. Alternatively, ion abundance can be calculated based on peak heights instead of peak areas.

Peak areas of all identified peptides from a given protein are added together to define a reconstructed peak area for the protein (step 360). Alternatively, the peak area for each identified peptide or polypeptide can be compared directly to the reference sample.

The relative quantity of a given protein in the experimental sample is determined by calculating the ratio of peak areas for the peptides or proteins in the experimental and reference samples (step 370). The reference sample can be a peptide mixture derived from a protein or mixture of proteins. In some implementations, the reference sample is expected to contain the protein or proteins for which quantitation information is desired. For example, the reference sample can be a mixture of proteins (e.g., cell samples, tissue samples, bodily fluids, etc.) taken from a known source (e.g., a healthy subject), while the experimental sample can be a similar mixture taken from an unknown source (e.g., a diseased subject). In one embodiment, the experimental sample and the reference sample are substantially similar, for example a plasma sample from a healthy living subject and a plasma sample from a deceased subject, and are expected to differ by only a small number of proteins. The peak areas for the reference sample can be derived from a sequence analogous to that illustrated in FIG. 3 and described above - i.e., digestion of the reference sample, separation of the protein digest, mass analysis, peptide identification, and chromatogram reconstruction to determine peak areas for peptides and proteins for the reference sample.

Method 300 can be repeated multiple (N) times to provide for relative quantitation for multiple samples, utilizing less than N references. Thus, for example, protein mixtures taken under a variety of conditions can be subjected to the techniques described herein to determine relative quantitation of proteins under those conditions.

Peak areas obtained for peptides in the same sample can differ from one run to another. These differences can be caused by a variety of experiment dependent parameters, such as differences in sample preparation (pipetting errors, incomplete digestion) or inaccurate sample injection. These experiment dependent parameters, while unknown in any given experiment, are expected to affect all proteins from a single run in the same way. The peak area thus calculated for each protein in the mixture can be normalized to correct for these systematic errors.

In some implementations, all peak areas can be normalized to the peak area of a known protein. The sample can include an internal standard. An internal standard can be one or more proteins that do not naturally occur in the sample and that are added to the sample to act as a reference for normalization—for example, a non-native protein that is added to the sample in a known amount. Alternatively, the internal standard can include a housekeeping protein or proteins - that is, a protein that is typically present in a relatively constant concentration in the medium from which the sample is derived. In such cases, the peak areas for each protein can be normalized to the peak area for the internal standard. Alternatively, the peak area for each protein can be normalized to the total peak area of all identified proteins in the mixture. To compare similar samples that differ only in the concentrations of a few proteins, such as cell cultures that are treated with different drugs, the peak areas or the ratios can be normalized against an obvious trend. For example, if the differences between the expected and the calculated peak areas for the proteins in a particular experiment are likely due to differences in sample preparation and are expected to affect all proteins from a single run in the same way, the peak areas can be normalized based on an average peak area ratio of all proteins that are constant over two or more experiments (or between the experimental and reference samples). Proteins that are present in different amounts in the different experiments (e.g., the proteins for which relative quantitation information is desired) can be excluded by calculating the standard deviation (e.g., the median standard deviation) of peak area ratios, excluding all proteins for which the ratio is are not within the median standard deviation, and recalculating the average (e.g., median) of the ratios for the remaining proteins. In one implementation, the standard deviation of the logarithmic values of the peak area ratios is calculated. In another implementation, the median of the ratios is used, because it is less susceptible to exceptions to the trend and is expected to be the best approach for a wide area of applications. Other known methods for normalizing the peak areas can also be used. The entire procedure can be repeated one or more times to increase precision of the relative quantitative measurements.

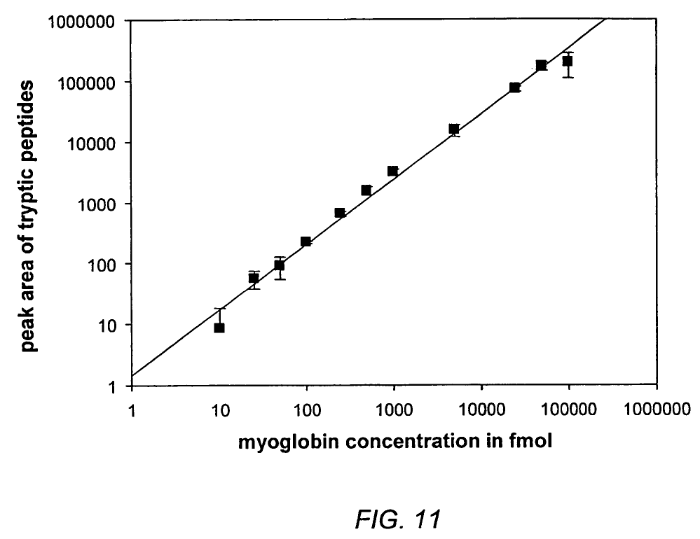

In another aspect of the invention, the relative quantitation of the peptides in an experimental sample can provide substantially absolute difference information since there is a linear correlation between the peak area of the peptides and its concentration. This is described in more detail in Example 3, Table 4 and FIG. 11.

Aspects of the invention can be implemented in digital electronic circuitry, or in computer hardware, firmware, software, or in combinations of them. Some or all aspects of the invention can be implemented as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Some or all of the method steps of the invention can be performed by one or more programmable processors executing a computer program to perform functions of the invention by operating on input data and generating output. Method steps can also be performed by, and apparatus of the invention can be implemented as, special purpose logic circuitry, e.g., an FPGA (field programmable gate array) or an ASIC (application-specific integrated circuit). The methods of the invention can be implemented as a combination of steps performed automatically, under computer control, and steps performed manually by a human user, such as a scientist.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

To provide for interaction with a user, the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well.

The invention will be further described in the following examples, which are illustrative only, and which are not intended to limit the scope of the invention described in the claims.

### EXAMPLES

### Example 1

The disclosed methods were applied to a mixture of five standard proteins—bovine albumin, horse hemoglobin, horse ferritin, horse cytochrome, and horse myoglobin. Four proteins were maintained at a constant concentration (200 fmol) while the concentration of the fifth protein (myoglobin) was varied over a wide range. Peak areas of protein digests were normalized to peak area of the albumin digest. The entire procedure was repeated three times. With 20% RSD after three measurements, the peak area calculated for the four constant-concentration protein digests was constant. The relative peak area of the fifth protein (myoglobin) showed a linear increase with increasing concentration from 10 fmol to 1000 fmol.

Sample Preparation

The five proteins were purchased from Sigma (St. Louis, Mo.) as lyophilized powder: bovine albumin, A-7638; horse hemoglobin, H-4632; horse ferritin, A-3641; horse myoglobin, M-0630; horse cytochrome C, C-7752. Solvents and reagents were purchased from different suppliers as following: acetonitrile, catalog # 015-1, Burdick & Jackson, Muskegon, Miss.; water, catalog # 4218-02, J T Backer, Phillipsburg, N.J.; formic acid, catalog # 11670, EM Science, Gibbstown, N.J.; ammonium bicarbonate, catalog # A-6141, Sigma; sequencing grade modified trypsin, catalog # V5113, Promega, Madison, Wis.; iodoacetic acid, catalog # 35603 and dithiothreitol (DTT), catalog # 20290, both from Pierce, Rockford, Ill..

Stock solutions of protein digests were prepared as follows. Each protein was dissolved in 100 mM ammonium bicarbonate buffer and reduced by adding DTT. Cysteine residues were carboxymethylated with iodoacetic acid prior to digestion with trypsin. The alkylation step increased the mass of cysteine residues by 58 Da. Stock solutions of the five protein digests were further diluted and mixed together to prepare a dilution series for myoglobin including 8 mixtures. 4-μl injected aliquots of these mixtures contained 1, 5, 10, 50, 100, 200, 500, and 1000 fmol of myoglobin. Albumin, hemoglobin, ferritin, and cytochrome C were present in every injected mixture at 200 fmol. The same stock solutions of five proteins were used to prepare a dilution series for cytochrome C also including 8 mixtures. In this series, injected amount of cytochrome C was different in each mixture and equal to 1, 5, 10, 50, 100, 200, 500, and 1000 fmol. In this series, concentrations of albumin, hemoglobin, ferritin, and myoglobin were constant and the injected amount of each of these proteins was 200 fmol. LC/MS/MS

A Surveyor HPLC system (Thenno Finnigan Corporation, San Jose, Calif.) included an autosampler and a high pressure pump. Eight 4-μl aliquots of the myoglobin dilution series and eight 4-μl aliquots of the cytochrome C dilution series were placed in wells of a 96-well plate with conical bottom (catalog # 249946, Nalge Nunc, Naperville, Ill.) covered with polyester sealing tape (catalog # 236366, Nalge Nunc) and inserted in the autosampler maintained at 4° C. All 16 samples were analyzed within one day according to the following procedure. The same sequence was repeated in three consecutive days, so every protein mixture from each dilution series was analyzed three times. A 4-μl aliquot of sample was aspirated from the bottom of the well into the autosampler needle and injected into a 20-μl sample loop. The rest of the loop was filled with a 0.1% solution of formic acid in water (“Solvent A”). In the autosampler needle and in the sample loop, the 4-μl aliquot of sample was sandwiched between two 1-μl bobbles of air. This so-called “no-waste injection” routine allowed complete injection of small amounts of sample. After injection, the autosampler valve switched and sample from the loop was loaded directly on a 75 μm ID×10 cm capillary HPLC column with 15 μm electrospray tip packed with BioBasic C 18 stationary phase, 5 μpm particles, 300A pore (New Objective, Inc., Cambridge, Mass.). The capillary column was loaded with 2 μl/min isocratic flow of Solvent A. For gradient elution, the 50 μl/min flow from the pump was split to 0.1 μl/min flow through the column. Peptides were eluted from the column with a linear gradient 0- 60% of a 0.1% solution of formic acid in acetonitrile (“Solvent B”). Eluting peptides were analyzed by a LCQ DECA ion trap mass spectrometer equipped with a nano-electrospray ion source (both Thermo Finnigan, San Jose, Calif.). The mass spectrometer operated in a data-dependent LC/MS/MS mode, in which the precursor ion was selected from the previous full-scan mass spectrum. Collision-induced dissociation was performed on the selected ion and its m/z value was dynamically excluded for 1 min from further fragmentation. This feature of automated analysis provided assess to a large number of peptides eluting (and often co-eluting) during LC/MS/MS analysis of complex mixtures.

Tandem mass spectra were correlated using TurboSequest software with a database containing 4400 sequences of horse and bovine proteins downloaded from National Center for Biotechnology Information web page at http://www.ncbi.nlm.nih.gov/Database/index.html. Output files from the correlation analysis were further summarized using a unified score of the three correlation coefficients generated by TurboSequest algorithm (Score=(10000×DelCn²+Sp)×Xcorr) to produce a list of identified peptides and corresponding proteins.

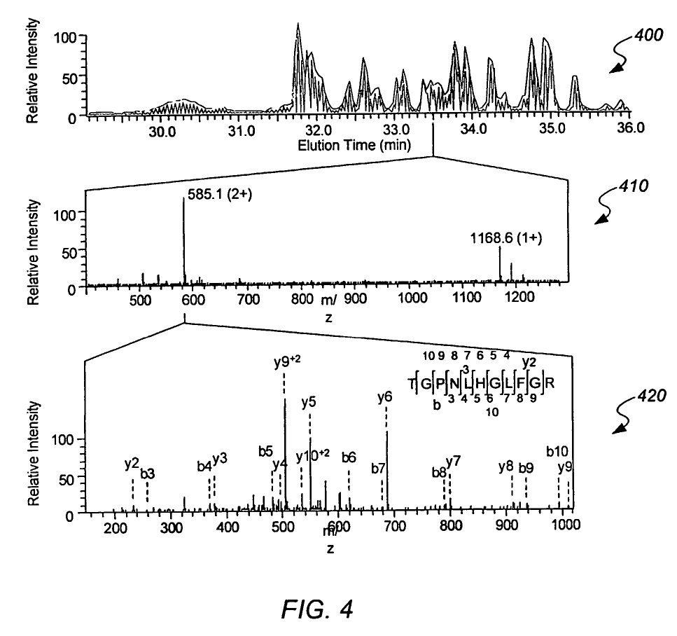

A typical ion chromatogram 400 of the five-protein digest mixture is shown in FIG. 4. In this mixture, all proteins were present at 200 fmol levels. During the LC/MS/MS analysis, a full-scan mass spectrum of eluting peptides was followed by a tandem mass spectrum creating a series of spikes on the chromatogram, in which the full scan mass spectra contributed to the top of the spikes. Whenever a single precursor peak was isolated and MS/MS was acquired, the ion current decreased creating a valley between two spikes. For quantitative peak area measurements, intensities of precursor ions from the full scan mass spectra were used—i.e. peaks on ion chromatogram were smoothed by a line drawn through the tops of the spikes as shown in FIG. 4. All identified digest products eluted in a 7-minute interval. Approximately 300 mass spectra, half of them MS and the other half MS/MS, were acquired during this period of time (i.e., 1.4 seconds per spectrum). Also shown in FIG. 4 are a full-scan MS 410 of digest products eluted at 33.50 minutes, as well as a MS/MS spectrum 420 of the precursor ion with m/z 585.1. The later mass spectrum is dominated by b and y types of fragments, which is a typical pattern for collision induced dissociation in an ion trap. Using TurboSequest software, the peak at m/z 585.1 was identified as the 2+ion of cytochrome C peptide TGPNLHGLFGR (SEQ ID NO:25). The peak at m/z 1168.6 was chosen for fragmentation during the next MS/MS scan and was identified as a singly charged ion of the same peptide, confirming the identification.

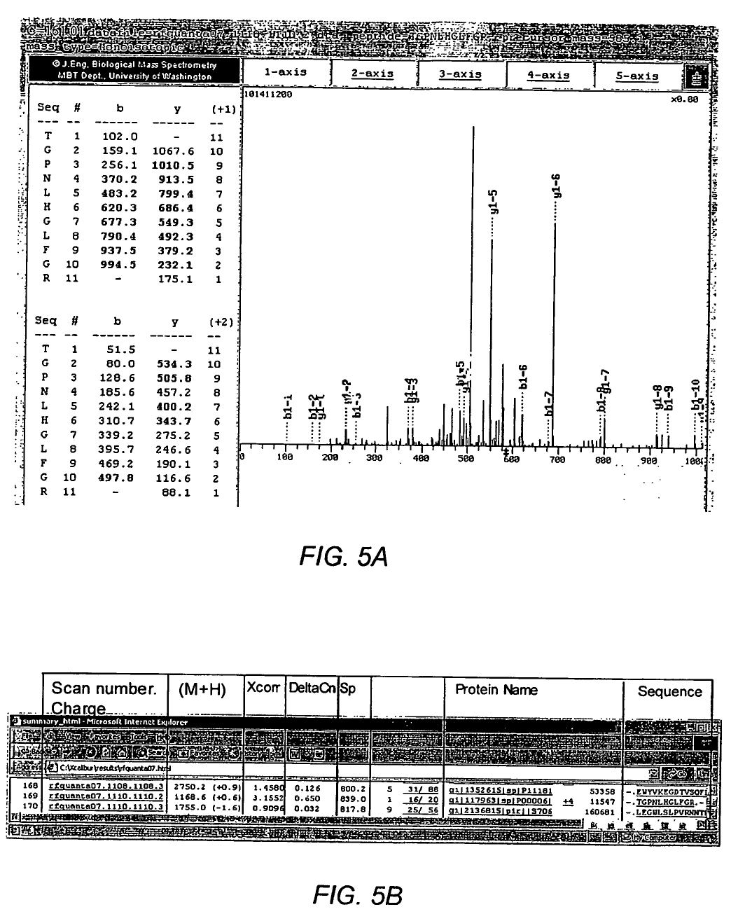

An example of a typical fragmentation mass spectrum and its interpretation, which is done automatically using TurboSequest software, is shown in FIG 5A. The software correlates the experimental fragmentation mass spectra with theoretical fragmentation patterns of all peptides from a protein database, and reports scan number; charge state; (M+H) value; three main correlation coefficients generated by TurboSequest (i.e., Xcorr, DeltaCn, Sp), protein name, identified sequence and several other parameters (FIG. 5B). These parameters are used to filter the true identifications from false.

LC/MS/MS analysis of the entire dilution series including the equimolar mixture in FIG. 4 was repeated three times. A total of 34 peptides were identified as digest products for the five-protein mixture, including 16 peptides from albumin, 7 peptides from hemoglobin, I peptide from ferritin, 3 peptides from cytochrome C, and 5 myoglobin peptides. Many of these peptides were represented by two or more charge forms. Every acquired tandem mass spectrum was correlated with the database three times under the assumption it could be produced from singly-, doubly-, or triply-charged precursor ions. Two charge forms of cytochrome C peptide TGPNLHGLFGR (SEQ ID NO:25) were subjected to collision induced dissociation during the elution time of this peptide adding extra confidence to the identification by TurboSequest. A total of 61 ions were identified as digest products for the five-protein mixture, or approximately 2 ion forms per each peptide. Table I lists the sequences of identified peptides, their charge nd m/z values, coefficients of cross correlation between each experimental spectrum and theoretical fragmentation pattern derived from the database, and of identified proteins with their gi numbers in NCBI database. All five proteins nambiguously identified in three different days. Only those peptides that were ied more than once were included in Table 1.

TABLE 1

| | | | | | | | |
|---|---|---|---|---|---|---|---|
| SEQ | | | | | | | |
| ID # | Peptide | Charge | m/z | Xcorr 1 | Xcorr2 | Xcorr3 | Protein |

| | | | | | | | |
|---|---|---|---|---|---|---|---|
| 1 | ALKAWSVAR | 2+ | 501.0 | 1.1 | 1.0 | | albumin, |
| 2 | EACFAVEGPK | 2+ | 555.0 | 2.7 | 2.2 | 2.1 | gi#2190337 |
| | | 1+ | 1108.5 | 1.0 | | 1.1 | |
| 3 | NECFLSHKDDSPDLPK | 3+ | 635.3 | 34. | 3.5 | | |
| | | 2+ | 952.1 | 4.1 | | | |
| 4 | CCAADDKEACFAVEGPK | 3+ | 644.8 | | 4.4 | 4.4 | |
| | | 2+ | 966.2 | 4.9 | 4.5 | 5.4 | |
| 5 | HLVDEPQNLIK | 2+ | 653.6 | 3.1 | 3.4 | | |
| | | 1+ | 1305.6 | 1.1 | 2.3 | 2.1 | |
| 6 | YNGVFQEGCQAEDK | 2+ | 875.6 | 4.1 | 3.8 | 2.8 | |
| 7 | YLYEIAR | 2+ | 464.7 | 2.7 | 2.3 | 2.7 | |
| | | 2+ | 927.5 | | | 1.5 | |
| 8 | DDPHACYSTVFDK | 3+ | 519.6 | 2.8 | 2.7 | 2.8 | |
| | | 2+ | 778.7 | 2.5 | 2.9 | 2.4 | |
| 9 | KVPQVSTPTLVEVSR | 3+ | 547.6 | 4.4 | 3.9 | 4.0 | |
| | | 2+ | 820.8 | 2.9 | 2.3 | 2.9 | |
| 10 | RHPEYAVSVLLR | 3+ | 481.0 | 4.2 | 4.1 | 3.8 | |
| | | 2+ | 720.8 | 2.9 | 2.3 | | |
| 11 | LKPDPNTLCDEFK | 3+ | 526.9 | 3.2 | 3.5 | 2.9 | |
| 12 | VPQVSTPTLVEVSR | 2+ | 756.7 | 3.3 | 3.0 | 3.3 | |
| 13 | KQTALVELLK | 2+ | 572.3 | 2.8 | 3.2 | 3.7 | |
| | | 1+ | 1142.5 | | 2.0 | | |
| 14 | LVNELTEFAK | 2+ | 582.6 | 3.6 | 3.3 | 3.5 | |
| | | 1+ | 1163.5 | 2.1 | 2.1 | | |
| 15 | SLHTLFGDELCK | 3+ | 474.7 | 3.1 | 3.1 | 3.5 | |
| | | 2+ | 711.0 | 3.2 | 3.1 | 3.5 | |
| | | 1+ | 1420.5 | 2.8 | | | |
| 16 | QTALVELLK | 2+ | 508.6 | 2.3 | | 2.2 | |
| | | 1+ | 1015.5 | | 1.2 | 1.3 | |
| 17 | VGGHAGEYGAEALER | 3+ | 505.7 | | | 3.1 | hemoglobin A, |
| | | 2+ | 757.8 | | 3.4 | | gi# 122411 and |
| 18 | DFTPELQASYQK | 2+ | 714.1 | 3.6 | 2.5 | 3.4 | hemoglobin B, |
| | | 1+ | 1426.6 | 2.0 | | 2.2 | gi# 122614 |
| 19 | TYFPHEDLSHGSAQVK | 3+ | 612.5 | 2.6 | | | |
| | | 2+ | 917.7 | 3.6 | 2.8 | | |
| 20 | FLSSVSTVLTSK | 2+ | 635.2 | 3.1 | 1.6 | 3.4 | |
| | | 1+ | 1268.6 | | | 1.4 | |
| 21 | AAVLALWDK | 2+ | 494.1 | 3.4 | 1.5 | 3.5 | |
| | | 1+ | 986.5 | 2.0 | 3.6 | 1.5 | |
| 22 | MFLGFPTTK | 2+ | 521.2 | 2.7 | 3.3 | 0.9 | |
| | | 1+ | 1041.5 | | 2.5 | 1.6 | |
| 23 | LLGNVLVVVLAR | 3+ | 423.1 | 4.2 | | | |
| | | 2+ | 633.5 | 3.8 | 3.3 | | |
| | | 1+ | 1265.9 | 1.2 | | | |
| 24 | QNYSTEVEAAVNR | 2+ | 741.2 | 4.1 | 4.3 | 2.3 | ferritin light |
| | | 1+ | 1480.7 | | 2.0 | | chain, gi# |
| | | | | | | | 1169741 |
| 25 | TGPNLHGLFGR | 2+ | 585.1 | 3.2 | 3.2 | 3.0 | ctochrome C, |
| | | 1+ | 1168.6 | 2.1 | 2.1 | 2.0 | gi# 117995 |
| 26 | MIFAGIK | 1+ | 779.5 | 1.7 | 1.5 | 1.6 | |
| 27 | EDLIAYLK | 2+ | 483 | 2.1 | 2.1 | 2.3 | |
| | 1+ | 964.5 | 2.0 | 1.8 | 1.9 | | |
| 28 | ELGFQG | 1+ | 650.2 | 1.0 | 1.1 | 1.2 | Myoglobin, gi# |
| 29 | YKELGFQG | 2+ | 471.7 | 2.7 | 3.5 | 2.7 | 0561 |
| | | 1+ | 941.4 | 1.8 | 1.7 | 2.0 | |
| 30 | VEADIAGHGQEVLIR | 3+ | 536.8 | 3.4 | 3.7 | 3.5 | |
| | | 2+ | 804.3 | 4.4 | 3.6 | 4.3 | |
| 31 | ALELFR | 1+ | 748.6 | | 1.0 | 1.1 | |
| 32 | HGTVVLTALGGILKK | 3+ | 503.4 | 4.0 | 4.2 | 4.2 | |
| 33 | HGTVVLTALGGILK | 3+ | 460.6 | 3.8 | 4.0 | 3.6 | |
| | | 2+ | 690.3 | 4.4 | 4.7 | 5.1 | |
| 34 | GLSDGEWQQVLNVWGK | 2+ | 908.9 | 4.8 | | | |

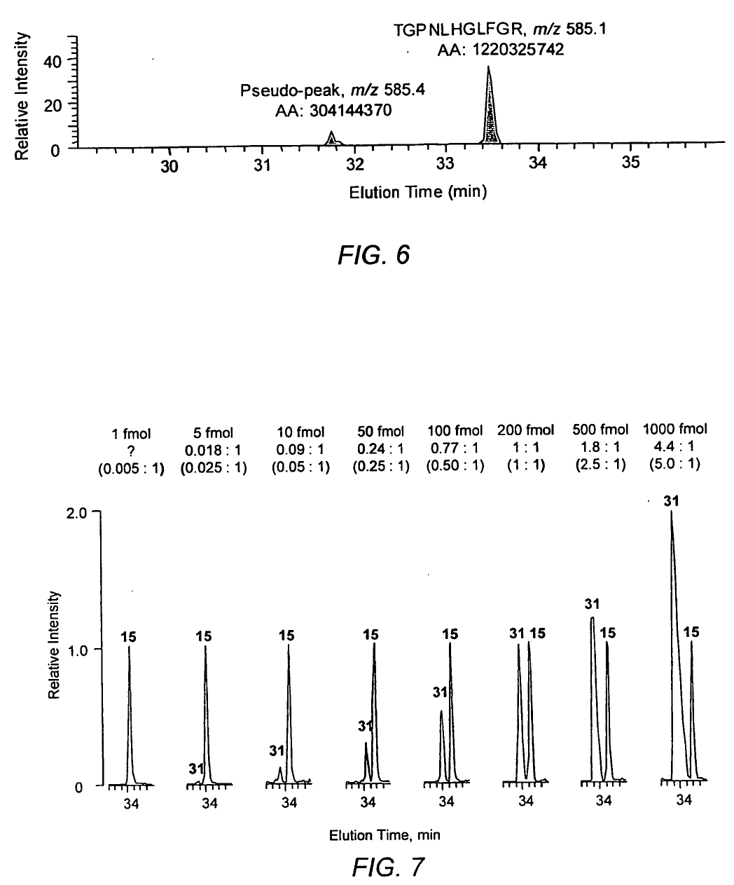

The chromatographic peak area of each identified ion was reconstructed using Xcalibur® software using the ion intensity from the corresponding full-scan mass spectrum. FIG. 6 is an example of such a reconstructed ion chromatogram for the 2+ion of the cytochrome C peptide TGPNLHGLFGR (SEQ ID NO:25). This reconstructed ion chromatogram was plotted using only intensities of mass spectral peaks with m/z 585.1±0.5. The automatically calculated peak area values (AA values) are shown in FIG. 6, where the peak area is reported in arbitrary units of ion intensity times seconds.

Although the true cytochrome C peptide eluted as a 0.2-min wide peak at 33.50 minutes, the chromatogram also features another, unidentified peak at 31.66 minutes. This pseudo-peak appeared on the reconstructed ion chromatogram, because its m/z value of 58.54 was close (within±0.5 Da) from the m/z value of the identified ion of cytochrome C. This pseudo-peak was excluded from consideration as follows. On average, the chromatographic peaks were 0.2 minute wide at the basement for our gradient of 0-60% B in 30 min (FIG. 6). Therefore, only the peaks located within ±0.2 minute on reconstructed ion chromatogram from the time of their identification were taken into account. This allowed for the removal of pseudo-peaks generated by species that were not the identified tryptic digest products but that had similar m/z values. The same rule was applied to other identified ions. This resulted in significant improvement in the precision of peak area measurements.

FIG. 7 illustrates eight reconstructed chromatograms for ions of the myoglobin peptide ALELFR (SEQ ID NO:31) with m/z 748.6 (1+) (number 31 in Table 1) and the albumin peptide SLHTLFGDELCK (SEQ ID NO: 15) with m/z 474.7 (3+), 711.0 (2+), and 1420.5 (1+) (number 15 in Table 1). Only a small, one-minute section of chromatogram was reconstructed near the elution time of 34 minutes, when both peaks elute. The albumin concentration was 200 fmol in all eight chromatograms, while the concentration of the myoglobin varied from 1 fmol to 100 fmol as illustrated. The reconstructed chromatographic peak area of the myoglobin peptide was observed to increase linearly with increasing myoglobin concentration and relative to albumin peptide at constant concentration. While the reconstructed chromatograms are illustrated in FIG. 7, no actual display of the reconstructed chromatogram and/or calculated peak areas is required.

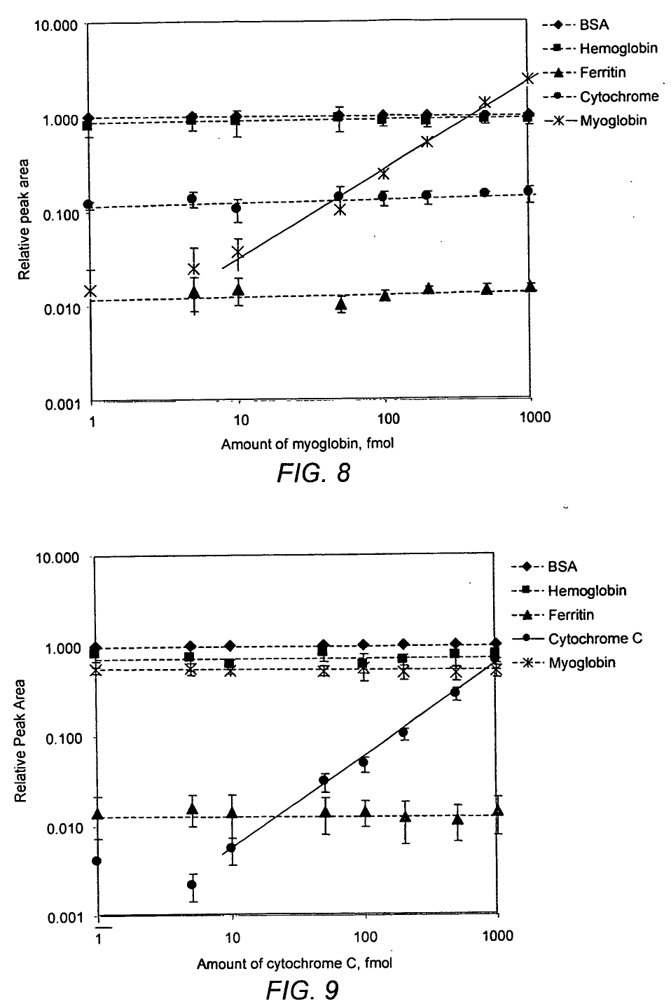

FIG. 8 illustrates a calibration curve for myoglobin digest (in amounts of 1, 5, 10, 50, 100, 200, 500, and 1000 fmol) mixed with constant amounts (200 fmol) of albumin, hemoglobin, ferritin, and cytochrome C. Plotted on the y axis are peak areas of protein digests for each protein normalized to peak area of albumin in each LC/MS/MS data file and averaged for three measurements in different days. Error bars show standard deviation (one sigma) of the measurements in three different days. Relative standard deviation (RSD) values for myoglobin at 1 and 5 fmol were above 60%, indicating that these measurements are at the noise level. RSD for 10 fmol was 36% and then fell below 15% for higher concentration in the dilution series, such that RSD values for the majority of data points on the plot are below 20%. The R2=0.9895 value for the linear trend line of myoglobin (not shown) indicates that the relative peak area of myoglobin digests increases linearly with increasing amounts from 10 fmol to 1000 fmol. For protein digests present in the mixture at constant level, reproducibility was also measured for 8 injections within each day and was better than 20% RSD.

The same set of 24 LC/MS/MS analyses and calculations was repeated for the five-protein mixture, varying the amount of cytochrome C in amounts of 1, 5, 10, 50, 100, 200, 500, and 1000 fmol and holding albumin, hemoglobin, ferritin, and myoglobin digests constant at 200 fmol. The series of 8 LC/MS/MS analyses was repeated three times in different days. FIG. 9 gives the calibration curve for cytochrome C. In FIG. 9, each data point is an average of three measurements. As in the myoglobin series, the RSD for cytochrome C data points at 1 and 5 fmol was very high, indicating that these concentrations could not be measured reproducibly. The data point at 10 fmol has 33% RSD and then reproducibility improves to below 20% RSD. R2=0.994 was the parameter value of the linear trend line for the cytochrome C (not shown) calibration curve.

### Example 2

Lypholized protein samples (1 mg human serum, and 1 mg horse myoglobin, Sigma-Aldrich, St. Louis, Mo., USA) were reconstituted in 1 ml of ammonium bicarbonate buffer (100 mM pH 8.5) and 3 μl DTT (1 M, Sigma-Aldrich, St. Louis, Mo., USA). The mixture was incubated for 30 minutes at 37° C. To alkylate the protein, 7 μl of iodoacetic acid (1 M in 1M KOH, Sigma-Aldrich, St. Louis, Mo., USA) was added and the mixture was incubated for an additional 30 minutes at room temperature in the dark. Thirteen μl DTT (I M) was added to quench the iodoacetic acid The reduced and alkylated proteins were digested by adding 20 μl trypsin (0.5 mg/ml, Promega, Madison, Wis., USA). The mixture was incubated for 6 hours at 37° C., then an additional 20 μl trypsin (0.5 mg/ml) was added and incubation was continued for 16 hours at 37° C.

Aliquots (as indicated in the text) of the sample digests were placed in wells of a 96-well plate. The plate was sealed with plastic film to minimize evaporation and positioned in the Surveyor auto-sampler, where it was maintained at 4° C. while waiting for analysis. The Surveyor auto-sampler was equipped with no-waste injection capability, which enables injection volumes as low as 1 μL. The injected peptides were first loaded on a small reversed-phase peptide trap poly (styrene-divinylbenzene) (Michrom Bioresources) with a relatively high flow rate of 10 μL/min for 3 minutes. Then peptides were eluted from the trap and subsequently separated on a reverse phase capillary column (PicoFrit; 5 μm BioBasic C18, 300 A pore size; 75 μm×10 cm; tip 15 μm, New Objective) with a 30-min linear gradient of 0-60% acetonitrile in 0.1% aqueous formic acid at a flow rate of 0.1 μL /min after split. The Surveyor HPLC system was directly coupled to a ThermoFinnigan LCQ Deca XP ion trap mass spectrometer equipped with a nano-LC electrospray ionization source. The spray voltage was 2.0 kV, the capillary temperature was 150° C. and ion-trap collision fragmentation spectra were obtained by collision energies of 35 units. Each full mass spectrum was followed by three MS/MS spectra of the three most intense peaks. The Dynamic Exclusion was enabled. After each sample an injection of 10 μL 0.1% aqueous formic acid was analyzed to ensure proper equilibration of the system.

Peptides and proteins were identified automatically by the computer program Sequest, which correlates the experimental tandem mass spectra against theoretical tandem mass spectra from amino acid sequences obtained from the National Center for Biotechnology Information (NCBI) sequence database. Peptide identification was further evaluated using a unified score combining all three correlation coefficients generated by Sequest. The score was calculated according to the following formula: Score=(10000×DelCn²+Sp)×Xcorr. For proteins the score of each peptide was added and the normalized score was calculated to be the total score divided by the numbers of peptides. Only peptides with a score of more than 2000 were accepted. The Genesis algorithm in the Xcalibur software was used for peak detection and calculation of the peak area.

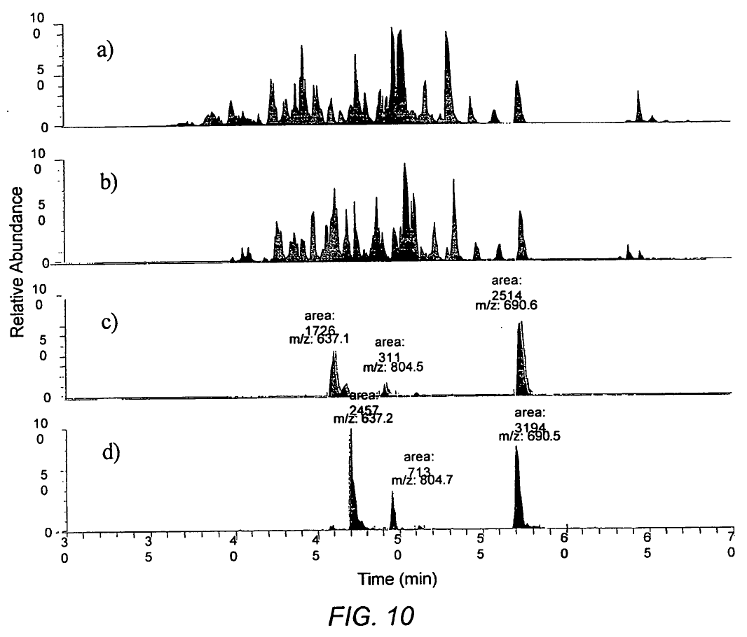

To further evaluate the quantitation method for protein profiling of complex mixtures human serum (approximately 1 μg total protein) was mixed with different amounts of horse myoglobin (250 fmol and 500 fmol) and the two mixtures were analyzed. Tryptic peptides were separated on a C-18 column with a gradient of 0-60% acetonitrile in 30 minutes. The chromatograms are shown in FIG. 10. Fragmentation information from MS/MS spectra and the automated search program Sequest was used for peptide and protein identification. A summary of all identified proteins is shown in Table 2. A total of 56 peptides corresponding to 20 different proteins could be identified in both samples. The same proteins were identified in both samples with only minor differences in peptide coverage (data not shown). The very low number of peptide and therefore proteins identified in this study is not surprising considering the amount of protein injected and the gradient used for peptide separation. The focus of this study was not to identify the maximum number of peptides in the sample rather than to ensure elution of all peptides in a small period of time. In similar experiments using longer gradients of up to 8 hours and using more material over 300 proteins could be identified.

For quantitative analysis a total of 16 peptides were chosen from 6 different proteins including 5 proteins from human serum (serum albumin, serotransferrin, alpha-I-antitrypsin, Ig gamma-4 chain C region and apolipoprotein A-1) and horse myoglobin. All proteins with more than one peptide identified were included in the quantitative analysis. The peak areas of these peptides were calculated as described above and the two samples were compared. The only difference in the two samples was the concentration of the horse myoglobin. In theory the peak area of the human proteins should be constant and only the peak area of the horse myoglobin should change.

The result of this experiment is summarized in Table 3. Comparison of sample 1 (250 fmol myoglobin) and sample 2 (500 fmol myoglobin) shows that the peak areas of the human peptides of sample 2 are all approximately the same or smaller (ratio from 1.04 to 0.69) whereas the myoglobin peptides are all higher (ratio from 1.27 to 2.29). The ratios of the peak areas were normalized against an experiment-dependent correction factor. This correction factor was calculated by excluding all ratios not within the median (0.92)±the standard deviation (0.42). The average of the remaining ratios was calculated to be 0.87 and all peak area ratio were normalized against this factor. The concentration of the human proteins was constant and therefore the peak areas should have a ratio of 1. Serum albumin was calculated to have a ratio of 0.91, serotransferrin was calculated to be 1.05, antitrypsin was calculated to be 0.84, Ig gamma-4 chain C region was calculated to be 0.95 and apolipoprotein A-I was calculated to be 1.10. The concentration of myoglobin in the second sample was double the concentration of myoglobin in the first sample and therefore the ratio of the peak areas should be 2. And indeed the peak area for horse myoglobin was calculated to be 1.91. The calculated ratio of the peak areas and the expected ratio of the peak areas are within 16% for the calculated proteins. The results confirm that peak area from peptides can be used for quantitative profiling of proteins in complex mixtures. This method can be used to detect small changes in protein concentrations from one sample to the other and gives information about the ratio at which the changes occur.

TABLE 2

| Protein | Peptides | Scans | Score | Norm. score |
|---|---|---|---|---|
| Serum albumin | 22 | 34 | 270 | 7955 |
| | | | 459 | |
| Serotransferrin | 8 | 12 | 98 574 | 8 214 |
| Myoglobin (horse) | 4 | 6 | 69 433 | 11 572 |
| Alpha-1-antitrypsin | 3 | 4 | 26 549 | 6 637 |
| Ig gamma-4 chain C region | 3 | 4 | 227 | 5 688 |
| | | | 511 | |
| Ig lambda chain C region | 1 | 2 | 21 148 | 10 574 |
| Ig gamma-1 chain C region | 1 | 2 | 15 492 | 7 746 |
| Apolipoprotein A-1 | 2 | 4 | 13 075 | 3 269 |
| Fibrinogen beta chain | 1 | 1 | 12 118 | 12 118 |
| Transthyretin | 1 | 2 | 10 070 | 3 035 |
| Haptoglobulin-2 | 1 | 1 | 9 725 | 9 725 |
| Ig alpha-1 chain C region | 1 | 2 | 8 588 | 4 294 |
| Fibrinogen gamma chain | 1 | 2 | 6 595 | 3 297 |
| Alpha-1 acid glycoprotein 2 | 1 | 1 | 5 821 | 5 821 |
| Ran binding protein 2 | 1 | 1 | 3 751 | 3 751 |
| Eukariotic translation initiation | 1 | 1 | 3 071 | 3 071 |
| factor 3 subunit 2 | | | | |
| Haptoglobulin-related protein | 1 | 1 | 2 848 | 2 848 |
| Transcription factor RELB | 1 | 1 | 2 782 | 2 782 |
| Serine/threonine protein | 1 | 1 | 2 500 | 2 500 |
| phosphatase 2B catalytic | | | | |
| subunit, beta isoform | | | | |
| S100 calcium-binding protein | 1 | 1 | 2 376 | 2 376 |
| A14 | | | | |

TABLE 3

| | | | | | | |
|---|---|---|---|---|---|---|
| | Peptides | Observed | Mean ± | NL | Expected | |
| Protein | identified | ratio | SD | ratio | ratio | % error |

| | | | | | | |
|---|---|---|---|---|---|---|
| Albumin | LCTVATLR | 0.87 | 0.79 ± 0.18 | 0.91 | 1 | 9 |
| | (SEQ ID NO:35) | | | | | |
| | YICENQDSISSK | 0.69 | | | | |
| | (SEQ ID NO:36) | | | | | |
| | CCAAADPHECYAK | 0.93 | | | | |
| | (SEQ ID NO:37) | | | | | |
| | KVPQVSTPTLVEVST | 0.72 | | | | |
| | (SEQ LD NO:38) | | | | | |
| Transferrin | DGAGDVAFVK | 0.85 | 0.91 ± 0.11 | 1.05 | 1 | 5 |
| | (SEQ ID NO:39) | | | | | |
| | SVIPSDGPSVACVK | 0.98 | | | | |
| | (SEQ ID NO:40) | | | | | |
| Antitrypsin | SVLGQLGITK | 0.76 | 0.73 ± 0.03 | 0.84 | 1 | 16 |
| | (SEQ ID NO:41) | | | | | |
| | LSITGTYDLK | 0.70 | | | | |
| | (SEQ ID NO:42) | | | | | |
| Myoglobin | HGTVVLTALGGILK | 1.27 | 1.66 ± 0.55 | 1.91 | 2 | 5 |
| | (SEQ ID NO:33) | | | | | |
| | VEADIAGHGQEVLIR | 2.29 | | | | |
| | (SEQ ID NO:30) | | | | | |
| | LFTGHPETLEK | 1.42 | | | | |
| | (SEQ ID NO:43) | | | | | |
| IgG-4 | GPSVFPLAPCSR | 0.62 | 0.83 ± 0.11 | 0.95 | 1 | 5 |
| | (SEQ ID NO:44) | | | | | |
| | NQVSLTGLVK | 1.04 | | | | |
| | (SEQ ID NO:45) | | | | | |
| Apo-A1 | THLAPYSDELR | 0.92 | 0.96 ± 0.04 | 1.10 | 1 | 10 |
| | (SEQ ID NO:46) | | | | | |
| | ATEHLSTLSEK | 1.00 | | | | |
| | (SEQ ID NO:47) | | | | | |

### Example 3

Eleven aliquots containing different amounts of myoglobin digests in the range from 10 fmol to 100 pmol were analyzed by LC/MS/MS, and the peak area of five selected peptides were calculated. The experiment was repeated three times to ensure repeatability. The peak area increases with increased concentration of injected peptides. In this experiment, the lower limit for peak detection was 10 fmol. The upper limit was 100 μpmol. The peak areas of all five myoglobin peptides were combined and plotted against the amount of myoglobin. The peak area correlates linear to the concentration of myoglobil (²=0.991) from 10 fmol to 100 pmol, and the results are repeatable. A summary of the results is shown in Table 4 and FIG. 11. It should be noted that the peak areas with a value 0 (see Table 4) could not be shown at the logarithmic scale but are included in the linear regression.

TABLE 4
ESI-MS Analysis of Myoglobin Proteolytic
Fragments from Tryptic Digestion of Horse Myoglobin

| Concn | Peak | Peak | Peak | | | % |
|---|---|---|---|---|---|---|
| (fmol) | Area 1 | II | III | Avg | SD | error |
| 100 000 | 272 819 | 105 719 | 199 122 | 192 886 | 84 223 | 44.0 |
| 50 000 | 170 712 | 144 559 | 194 372 | 169 881 | 24 917 | 15.0 |
| 25 000 | 67 095 | 70 790 | 81 044 | 72 976 | 7 227 | 9.9 |
| 5 000 | 12 820 | 13 879 | 19 128 | 15 275 | 3 378 | 22.0 |
| 1 000 | 3 492 | 3 224 | 2 768 | 3 161 | 366 | 12.0 |
| 500 | 1 289 | 1 651 | 1 764 | 1 568 | 248 | 16.0 |
| 250 | 714 | 643 | 588 | 648 | 63 | 9.7 |
| 100 | 212 | 219 | 231 | 221 | 9.6 | 4.4 |
| 50 | 130 | 97 | 61 | 90 | 36 | 40.0 |
| 25 | 38 | 74 | 55 | 56 | 18 | 32.0 |
| 10 | 19 | 0 | 6 | 8.3 | 9.7 | 117.0 |
| 0 | 0 | 0 | 0 | 0 | 0 | 0 |

The invention has been described in terms of particular embodiments. Other embodiments are within the scope of the following claims. For example, the steps of the invention can be performed in a different order, and/or combined, and still achieve desirable results.

In addition, the invention has been described in terms of embodiments relating to peptides, polypeptides and proteins, whether naturally occurring, synthetic or otherwise created. It will be apparent that the techiques described herein may also be applied to other materials, for example fatty acids, DNAs, RNAs, digonucleotides, organic or inorganic molecules, etc.

What is claimed is:

1-43. (canceled)

44. A method for quantifying one or more peptides in a peptide mixture, comprising:
receiving a first peptide mixture containing a plurality of peptides;
separating one or more of the plurality of peptides of the first peptide mixture over a period of time;
mass-to-charge analyzing one or more of the separated peptides of the first peptide mixture at a particular time in the period of time;
calculating an abundance of one or more of the mass analyzed peptides of the first peptide mixture; and
calculating a relative quantity for the one or more mass analyzed peptides of the first peptide mixture by comparing the calculated abundance of the one or more mass analyzed peptides of the first peptide mixture with an abundance of one or more peptides in a reference sample, the reference sample being external to the first peptide mixture.

45. The method of claim 44, wherein:
receiving a first peptide mixture containing a plurality of peptides comprises digesting a first polypeptide sample to generate the first peptide mixture.

46. The method of claim 45, further comprising:
preparing the reference sample by digesting a second polypeptide sample;
separating one or more peptides from the digested second polypeptide sample;
mass analyzing the separated peptides from the digested second polypeptide sample; and
calculating an abundance of one or more of the mass analyzed peptides from the second polypeptide sample;
wherein calculating a relative quantity for the one or more mass analyzed peptides of the first peptide mixture comprises comparing the calculated abundance of the one or more mass analyzed peptides of the first peptide mixture with the calculated abundance of one or more corresponding mass analyzed peptides from the second polypeptide sample.

47. The method of claim 44, wherein:
separating one or more peptides comprises separating the one or more peptides by liquid chromatography.

48. The method of claim 47, wherein:
separating one or more peptides comprises isolating a liquid chromatography eluent at the particular time; and
mass analyzing one or more of the separated peptides of the first peptide mixture comprises mass analyzing one or more peptides in the isolated eluent.

49. The method of claim 44, further comprising:
identifying one or more peptides of the first peptide mixture.

50. The method of claim 49, wherein:
identifying one or more peptides of the first peptide mixture comprises identifying one or more of the separated peptides based on mass analysis information.

51. The method of claim 50, wherein:
mass analyzing one or more of the separated peptides comprises fragmenting an ion derived from a peptide of the one or more separated peptides and mass analyzing fragments of the ion; and
identifying one or more peptides in the first sample comprises searching a sequence database based on mass analysis information for the fragments.

52. The method of claim 47, wherein:
calculating an abundance of one or more of the mass analyzed peptides comprises reconstructing a chromatogram peak for a peptide based on mass analysis information for the peptide.

53. The method of claim 52, wherein:
calculating an abundance for a peptide comprises calculating an abundance for a peptide based on a reconstructed chromatogram peak area for the peptide.

54. The method of claim 53, wherein:
calculating the abundance for a peptide comprises calculating an abundance for a peptide using only chromatogram peaks located within a threshold distance in the reconstructed chromatogram of the particular time.

55. The method of claim 53, wherein:
calculating a relative quantity for the one or more mass analyzed peptides comprises comparing an abundance calculated by reconstructing a chromatogram peak area for a peptide of the first peptide mixture with an abundance calculated by reconstructing a chromatogram peak area for a peptide in the reference sample.

56. The method of claim 45, further comprising:
normalizing the calculated abundance of the one or more mass analyzed peptides of the first peptide mixture.

57. The method of claim 56, wherein:
normalizing the calculated abundance comprises normalizing the calculated abundance based on an internal standard including one or more peptides added to the first polypeptide sample.

58. The method of claim 56, wherein:
normalizing the calculated abundance comprises normalizing the calculated abundance based on an external standard including one or more peptides.

59. The method of claim 45, further comprising:
identifying a plurality of peptides of the first peptide mixture based on the mass analyzing;
wherein calculating a relative quantity for the one or more mass analyzed peptides comprises calculating a relative quantity for each of the identified peptides.

60. The method of claim 59, further comprising:
normalizing calculated abundances for each of the identified peptides by calculating a correction factor based on reconstructed chromatogram peak areas for a set of peptides in the first peptide mixture, each peptide in the set of peptides having constant chromatogram peak areas over a plurality of experiments, and applying the correction factor to the calculated abundance for each of the identified peptides.

61. The method of claim 44, wherein:
mass-to-charge analyzing one or more of the separated peptides and calculating an abundance of one or more of the mass analyzed peptides comprises mass-to-charge analyzing and calculating an abundance for one or more arbitrary peptides of the first peptide mixture.

62. A method of quantifying one or more peptides in a mixture, comprising:
digesting a protein sample to generate a mixture of peptides;
separating one or more peptides of the mixture of peptides using liquid chromatography;
mass analyzing one or more of the separated peptides;
identifying one or more of the mass analyzed peptides based on mass spectra for the peptides;
calculating chromatogram peak areas for the identified peptides;
calculating chromatogram peak areas for one or more proteins corresponding to the identified peptides based on the calculated peak areas for the corresponding peptides;
normalizing the chromatogram peak area for the protein based on a chromatogram peak area for an internal standard; and
determining a relative quantity for a protein of the one or more of the proteins by comparing the normalized chromatogram peak area for the protein to a chromatogram peak area for a corresponding protein in a reference sample.

63. An apparatus for quantifying one or more peptides in a peptide mixture, comprising:
means for receiving a first peptide mixture containing a plurality of peptides;
means for separating one or more of the plurality of peptides of the first peptide mixture over a period of time;
means for mass analyzing one or more of the separated peptides of the first peptide mixture at a particular time in the period of time;
means for calculating an abundance of one or more of the mass analyzed peptides of the first peptide mixture;
means for calculating a relative quantity for the one or more mass analyzed peptides of the first peptide mixture by comparing the calculated abundance of the one or more mass analyzed peptides of the first peptide mixture with an abundance of one or more peptides in a reference sample which is external to first peptide mixture.

64. The apparatus of claim 63, further comprising:
means for receiving at least one additional peptide mixture.

65. The apparatus of claim 64, wherein:
the at least one additional peptide mixture comprises a reference sample.

66. The apparatus of claim 63, wherein:
the means for calculating an abundance further comprises reference information.

67. The apparatus of claim 63, wherein:
the means for mass-to-charge analyzing and the means for calculating are configured to mass-to-charge analyze and calculate an abundance for one or more arbitrary peptides of the first peptide mixture.

68. The apparatus of claim 63, wherein:
the means for separating, mass-to-charge analyzing, and calculating steps are configured to separate, mass-to-charge analyze and calculate an abundance for one or more peptides independent of a particular amino acid composition of the subject peptides.

69. A computer program product on a computer-readable medium for quantifying one or more peptides in a first peptide mixture, the product comprising instructions operable to cause a programmable processor to:
receive separation information representing a separation of one or more of a plurality of peptides of a first peptide mixture over a period of time;
receive mass-to-charge analysis information for one or more of the separated peptides of the first peptide mixture at a particular time in the period of time;
calculate an abundance of one or more of the mass analyzed peptides of the first peptide mixture; and
calculate a relative quantity for the one or more mass analyzed peptides of the first peptide mixture by comparing the calculated abundance of the one or more mass analyzed peptides of the first peptide mixture with an abundance of one or more peptides in a reference sample, the reference sample being external to the first peptide mixture.

70. A computer program product on a computer-readable medium for quantifying one or more peptides in a first peptide mixture, the product comprising instructions operable to cause a programmable processor to:
receive separation information representing a separation of one or more of a plurality of peptides of a first peptide mixture over a period of time;
receive mass-to-charge analysis information for one or more of the separated peptides of the first peptide mixture at a particular time in the period of time;
identify one or more of the mass analyzed peptides based on the mass-to-charge analysis information for the peptides;
calculate chromatogram peak areas for the identified peptides;
calculate chromatogram peak areas for one or more proteins corresponding to the identified peptides based on the calculated peak areas for the corresponding peptides;
normalize the chromatogram peak area for the protein based on a chromatogram peak area for an internal standard; and
determine a relative quantity for a protein of the one or more of the proteins by comparing the normalized chromatogram peak area for the protein to a chromatogram peak area for a corresponding protein in a reference sample.

71. Apparatus for quantifying one or more peptides in a first peptide mixture, the apparatus comprising digital circuitry configured to perform the following actions:
receive separation information representing a separation of one or more of a plurality of peptides of a first peptide mixture over a period of time;
receive mass-to-charge analysis information for one or more of the separated peptides of the first peptide mixture at a particular time in the period of time;
calculate an abundance of one or more of the mass analyzed peptides of the first peptide mixture; and
calculate a relative quantity for the one or more mass analyzed peptides of the first peptide mixture by comparing the calculated abundance of the one or more mass analyzed peptides of the first peptide mixture with an abundance of one or more peptides in a reference sample, the reference sample being external to the first peptide mixture.

72. Apparatus for quantifying one or more peptides in a first peptide mixture, the apparatus comprising digital circuitry configured to perform the following actions:
receive separation information representing a separation of one or more of a plurality of peptides of a first peptide mixture over a period of time;
receive mass-to-charge analysis information for one or more of the separated peptides of the first peptide mixture at a particular time in the period of time;
identify one or more of the mass analyzed peptides based on the mass-to-charge analysis information for the peptides;
calculate chromatogram peak areas for the identified peptides;
calculate chromatogram peak areas for one or more proteins corresponding to the identified peptides based on the calculated peak areas for the corresponding peptides;
normalize the chromatogram peak area for the protein based on a chromatogram peak area for an internal standard; and
determine a relative quantity for a protein of the one or more of the proteins by comparing the normalized chromatogram peak area for the protein to a chromatogram peak area for a corresponding protein in a reference sample.

73. A method for quantifying one or more compounds in a biological sample, comprising:
receiving a biological sample containing a plurality of compounds;
separating one or more of the plurality of compounds of the biological sample over a period of time;
mass-to-charge analyzing one or more of the separated compounds of the biological sample at a particular time in the period of time;
calculating an abundance of one or more of the mass analyzed compounds of the biological sample; and
calculating a relative quantity for the one or more mass analyzed compounds of the biological sample by comparing the calculated abundance of the one or more mass analyzed compounds of the biological sample with an abundance of one or more compounds in a reference sample, the reference sample being external to the biological sample.

74. Apparatus for quantifying one or more compounds in a biological sample, the apparatus comprising digital circuitry configured to perform the following actions:
receive a biological sample containing a plurality of compounds;
separate one or more of the plurality of compounds of the biological sample over a period of time;
mass-to-charge analyze one or more of the separated compounds of the biological sample at a particular time in the period of time;
calculate an abundance of one or more of the mass analyzed compounds of the biological sample; and
calculate a relative quantity for the one or more mass analyzed compounds of the biological sample by comparing the calculated abundance of the one or more mass analyzed compounds of the biological sample with an abundance of one or more compounds in a reference sample, the reference sample being external to the biological sample.


SEQUENCE LISTING (47 sequences)

SEQ ID NO: 1 - protein, 9 aa, Bos taurus
ALKAWSVAR

SEQ ID NO: 2 - protein, 10 aa, Bos taurus
EACFAVEGPK

SEQ ID NO: 3 - protein, 16 aa, Bos taurus
NECFLSHKDDSPDLPK

SEQ ID NO: 4 - protein, 17 aa, Bos taurus
CCAADDKEACFAVEGPK

SEQ ID NO: 5 - protein, 11 aa, Bos taurus
HLVDEPQNLIK

SEQ ID NO: 6 - protein, 14 aa, Bos taurus
YNGVFQECCQAEDK

SEQ ID NO: 7 - protein, 7 aa, Bos taurus
YLYEIAR

SEQ ID NO: 8 - protein, 13 aa, Bos taurus
DDPHACYSTVFDK

SEQ ID NO: 9 - protein, 15 aa, Bos taurus
KVPQVSTPTLVEVSR

SEQ ID NO: 10 - protein, 12 aa, Bos taurus
RHPEYAVSVLLR

SEQ ID NO: 11 - protein, 13 aa, Bos taurus
LKPDPNTLCDEFK

SEQ ID NO: 12 - protein, 14 aa, Bos taurus
VPQVSTPTLVEVSR

SEQ ID NO: 13 - protein, 10 aa, Bos taurus
KQTALVELLK

SEQ ID NO: 14 - protein, 10 aa, Bos taurus
LVNELTEFAK

SEQ ID NO: 15 - protein, 12 aa, Bos taurus
SLHTLFGDELCK

SEQ ID NO: 16 - protein, 9 aa, Bos taurus
QTALVELLK

SEQ ID NO: 17 - protein, 15 aa, Equus caballus
VGGHAGEYGAEALER

SEQ ID NO: 18 - protein, 12 aa, Equus caballus
DFTPELQASYQK

SEQ ID NO: 19 - protein, 16 aa, Equus caballus
TYFPHFDLSHGSAQVK

SEQ ID NO: 20 - protein, 12 aa, Equus caballus
FLSSVSTVLTSK

SEQ ID NO: 21 - protein, 9 aa, Equus caballus
AAVLALWDK

SEQ ID NO: 22 - protein, 9 aa, Equus caballus
MFLGFPTTK

SEQ ID NO: 23 - protein, 12 aa, Equus caballus
LLGNVLVVVLAR

SEQ ID NO: 24 - protein, 13 aa, Equus caballus
QNYSTEVEAAVNR

SEQ ID NO: 25 - protein, 11 aa, Equus caballus
TGPNLHGLFGR

SEQ ID NO: 26 - protein, 7 aa, Equus caballus
MIFAGIK

SEQ ID NO: 27 - protein, 8 aa, Equus caballus
EDLIAYLK

SEQ ID NO: 28 - protein, 6 aa, Equus caballus
ELGFQG

SEQ ID NO: 29 - protein, 8 aa, Equus caballus
YKELGFQG

SEQ ID NO: 30 - protein, 15 aa, Equus caballus
VEADIAGHGQEVLIR

SEQ ID NO: 31 - protein, 6 aa, Equus caballus
ALELFR

SEQ ID NO: 32 - protein, 15 aa, Equus caballus
HGTVVLTALGGILKK

SEQ ID NO: 33 - protein, 14 aa, Equus caballus
HGTVVLTALGGILK

SEQ ID NO: 34 - protein, 16 aa, Equus caballus
GLSDGEWQQVLNVWGK

SEQ ID NO: 35 - protein, 8 aa, Homo sapiens
LCTVATLR

SEQ ID NO: 36 - protein, 12 aa, Homo sapiens
YICENQDSISSK

SEQ ID NO: 37 - protein, 13 aa, Homo sapiens
CCAAADPHECYAK

SEQ ID NO: 38 - protein, 15 aa, Homo sapiens
KVPQVSTPTLVEVST

SEQ ID NO: 39 - protein, 10 aa, Homo sapiens
DGAGDVAFVK

SEQ ID NO: 40 - protein, 14 aa, Homo sapiens
SVIPSDGPSVACVK

SEQ ID NO: 41 - protein, 10 aa, Homo sapiens
SVLGQLGITK

SEQ ID NO: 42 - protein, 10 aa, Homo sapiens
LSITGTYDLK

SEQ ID NO: 43 - protein, 11 aa, Equus caballus
LFTGHPETLEK

SEQ ID NO: 44 - protein, 12 aa, Homo sapiens
GPSVFPLAPCSR

SEQ ID NO: 45 - protein, 10 aa, Homo sapiens
NQVSLTCLVK

SEQ ID NO: 46 - protein, 11 aa, Homo sapiens
THLAPYSDELR

SEQ ID NO: 47 - protein, 11 aa, Homo sapiens
ATEHLSTLSEK